(19) United States
(12) Patent Application Publication
(10) Pub. No.: US 2024/0094201 A1
(43) Pub. Date: Mar. 21, 2024

(54) URCHIN-LIKE BEADS WITH ENHANCED OPTICAL PROPERTIES AND METHOD OF MAKING THEREOF

(71) Applicant: ALIGNEDBIO AB, SAN FRANCISCO, CA (US)

(72) Inventors: Niklas Mårtensson, Lund (SE); Jaime Castillo-León, Lund (SE); Søren Morgenthaler Echwald, Lund (SE)

(21) Appl. No.: 18/275,189

(22) PCT Filed: Feb. 4, 2022
(86) PCT No.: PCT/US22/15323

§ 371 Date: Jul. 31, 2023

Related U.S. Application Data
(60) Provisional application No. 63/146,088, filed on Feb. 5, 2021.

(51) Int. Cl. G01N 33/553; C08F 22/10; C08F 22/20; G01N 33/532; G01N 33/543

(52) U.S. Cl. CPC G01N 33/553; C08F 22/1006; C08F 22/20; G01N 33/532; G01N 33/54346; C08F 2410/06; G01N 2021/6432

(57) ABSTRACT

A method includes forming spherical liquid droplets of a first liquid phase containing a polymer precursor dispersed in a second liquid phase, where nanowires are located at an interface of the first and the second liquid phase and the nanowires extend with their longest axis substantially perpendicular to a surface of the spherical liquid droplets, and polymerizing the polymer precursor in the spherical liquid droplets to form beads including a solid polymer core and multiple nanowires aligned with their longest axis substantially perpendicular to a surface of the solid polymer core.

### RELATED APPLICATIONS

This application claims the benefit of priority of U.S. Non-Provisional Application No. 63/146,088, filed Feb. 5, 2021, the entire contents of which is herein incorporated by reference.

### FIELD

The present invention relates to an urchin-like solid bead and a method for producing the urchin-like beads. The fabricated urchin-like beads can be used as transducers in optical sensors.

### BACKGROUND

Conventional optical sensors, such as biosensors or chemical sensors (e.g., gas sensors, etc.), use sensor transducers, which may include microstructures, for optical sensing. The binding of an analyte on the sensor transducer is optically detected and output by the sensor as a display or other information about the analyte binding. Immunoassays currently utilize sensor transducer surfaces to immobilize sensing elements, such as enzymes or antibodies, that will bind analytes of interest.

### SUMMARY

According to various embodiments, a method includes forming spherical liquid droplets of a first liquid phase containing a polymer precursor, wherein the spherical liquid droplets are dispersed in a second liquid phase, and wherein nanowires are located at an interface of the first and the second liquid phase and the nanowires extend with their longest axis substantially perpendicular to a surface of the spherical liquid droplets, and polymerizing the polymer precursor in the spherical liquid droplets to form beads comprising a solid polymer core and multiple nanowires aligned with their longest axis substantially perpendicular to a surface of the solid polymer core.

According to various embodiments, a bead comprises a solid polymer core, and nanowires aligned with their longest axis substantially perpendicular to a surface of the solid polymer core. In one embodiment, the bead may be functionalized with at least one sensor element and used as a sensor transducer of an optical sensor which also includes a radiation source and a radiation detector.

According to various embodiments, a method comprises forming a dispersion comprising micelles dispersed in a second solvent, the micelles comprising droplets of a first solvent, a polymer precursor dispersed in the first solvent, and nanowires that radially extend from the droplets into the second solvent; and polymerizing the polymer precursor to form beads that comprise solid polymer cores and the nanowires radially extending from the solid polymer cores.

### BRIEF DESCRIPTION OF THE DRAWINGS

FIG. 1A is a schematic cross-sectional view of an urchin-like bead, according to various embodiments of the present disclosure, and FIG. 1B is a schematic cross-sectional view of a nanowire that may be included in the bead of FIG. 1A.

FIGS. 2A, 2B, and 2C are schematic side views of steps in making the urchin-like beads according to an embodiment of the present disclosure.

FIGS. 3A-3E are top cross-sectional views of microfluidic devices that may be used to form micelles, according to alternative embodiments of the present disclosure.

FIGS. 4A and 4B are optical micrographs of an urchin-like bead, according to various embodiments of the present disclosure.

FIGS. 5A, 5B, and 5C are scanning electron microscopy (SEM) images of urchin-like beads, according to various embodiments of the present disclosure.

FIG. 6 is a micrograph and chart showing the optical spectrum from an urchin-like bead, according to various embodiments of the present disclosure.

FIG. 7 is a bar graph comparing the spectral intensity from the urchin-like beads according to an embodiment of the present disclosure and to spectral intensity from polystyrene beads without nanowires.

### DETAILED DESCRIPTION

As set forth herein, various aspects of the disclosure are described with reference to the exemplary embodiments and/or the accompanying drawings in which exemplary embodiments of the invention are illustrated. This invention may, however, be embodied in many different forms and should not be construed as limited to the exemplary embodiments shown in the drawings or described herein. It will be appreciated that the various disclosed embodiments may involve particular features, elements or steps that are described in connection with that particular embodiment. It will also be appreciated that a particular feature, element or step, although described in relation to one particular embodiment, may be interchanged or combined with alternate embodiments in various non-illustrated combinations or permutations.

Ranges can be expressed herein as from “about” one particular value, and/or to “about” another particular value. When such a range is expressed, examples include from the one particular value and/or to the other particular value. Similarly, when values are expressed as approximations, by use of the antecedent “about” or “substantially” it will be understood that the particular value forms another aspect. In some embodiments, a value of “about X” may include values of +/−1% X. It will be further understood that the endpoints of each of the ranges are significant both in relation to the other endpoint, and independently of the other endpoint.

As referred to herein, a nanowire (also referred to as a nanorod or nanotube) has an aspect ratio of at least 10 (i.e., length greater than width by a factor of at least 10), such as 10 to 1,000. The nanowires 110 may have a length (i.e., longest axis dimension) of from 500 nm to 50 microns, such as from 2 to 40 microns, and a width (i.e., a dimension perpendicular to the longest axis, such as diameter for cylindrical nanowires) of from 5 nm to 1 micron, such as from 10 nm to 800 nm, of from 100 nm to 500 nm.

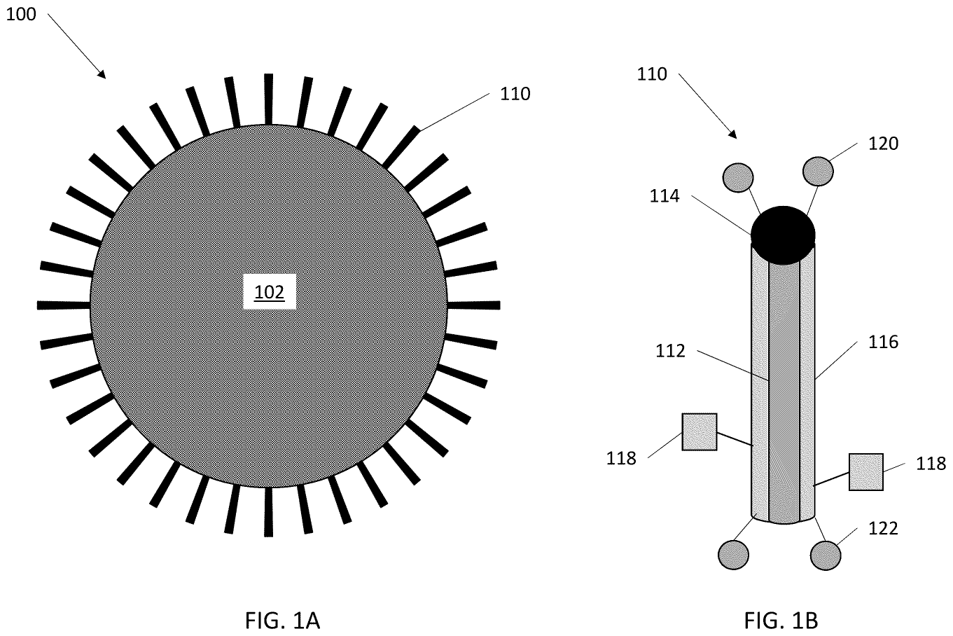

FIG. 1A is a cross-sectional view of an urchin-like bead 100 according to various embodiments of the present disclosure. FIG. 1B is a perspective view of a nanowire 110 that may be included in the bead of FIG. 1A. Referring to FIGS. 1A and 1B, the bead 100 may include a solid polymer core 102 and the nanowires 110 that may extend radially from the core 102 (e.g., are aligned with their longest axis substantially perpendicular to a corresponding portion of the surface of the core 102). The beads 100 may be used as sensor transducers in an optical sensor, such as a chemical sensor or a biosensor (e.g., immunoassay, etc.). The nanowires 110 may increase the surface area of beads 100, as compared to the surface area of the core 102.

In some embodiments, the nanowires 110 and/or the core 102 may be functionalized with fluorescent labels, such as fluorophores or quantum dots, which emit radiation when exposed to incident radiation, and/or with sensing elements configured to bind with specific analytes of interest. For example, the sensing elements may include one more enzymes, proteins, antibodies, or a combination thereof.

The nanowires 110 may be hollow nanowires (e.g., carbon nanotubes), solid nanowires, or a combination thereof. Examples of solid nanowires may include semiconductor nanowires, dielectric (i.e., electrically insulating) nanowires, or electrically conductive (e.g., metal) nanowires, or a combination thereof. Semiconductor nanowires may comprise elemental (e.g., Group IV) semiconductor nanowires, such as silicon or germanium nanowires, or compound semiconductor nanowires, such as Group IV-IV semiconductor nanowires (e.g., SiGe or SiC), Group III-V semiconductor nanowires or Group Il-VI semiconductor nanowires. Group III-V semiconductor nanowires include III-phosphide nanowires, such as InP, GaP or ternary or quaternary compounds thereof (e.g., AlGaP, InGaP, etc.), III-arsenide nanowires, such as GaAs, InAs or ternary or quaternary compounds thereof, or III-nitride nanowires, such as GaN or ternary or quaternary compounds thereof.

The nanowires 110 may each include a semiconductor wire 112 formed of a semiconductor material listed above, and an optional metal catalyst particle 114. The metal catalyst particle 114 may comprise Au, In, a Group III—gold alloy, such as an InAu alloy, or the like. Metal nanowires may comprise a metal, such as Ag, Au, Ga, In, Al, or their alloys. Dielectric nanowires may comprise silicon oxide, aluminum oxide, zinc oxide, or other metal oxide or ceramic nanowires. In one embodiment, the nanowires 110 may also comprise core-shell nanowires or nanowires with different compositions along the longest axis. For example, the core-shell nanowires may comprise a dielectric shell 116 that covers the semiconductor wire 112. The dielectric shell 116 may comprise silicon oxide or a metal oxide (e.g., alumina, etc.), and may have a thickness ranging from 10 nm to 40 nm. The metal catalyst particle 114 may be exposed outside of the shell 116.

The core 102 may comprise a polymer material, such as an acrylate polymer or the like. The core 102 may have a substantially spherical shape (e.g., shape of a perfect sphere or a sphere with less than 20% elongation along at least one axis compared to another axis). The core 102 may have longest dimension (e.g., diameter) of 10 microns to 1,000 microns. At least 50% of the nanowires 110, such as at least 75% of the nanowires 110, for example 80 to 100% of the nanowires 110 are axially aligned on each core 102, such that a long axis of each nanowire 110 is substantially perpendicular (e.g., within 70 to 110 degrees, such as 80 to 100 degrees, for example 90 degrees) to the bead core's surface. In other words, the nanowires 110 may extend in a direction that is within 20 degrees, such as within 10 degrees, or within about 5 degrees of a corresponding radial axis of the bead 102. The nanowires 110 may be straight or be somewhat bent, but with a predominant longest (e.g., lengthwise) axis still discernable.

The nanowires 110 may include detection elements 118. In one embodiment, the detection elements 118 may include optical elements that emit radiation when exposed to incident radiation, such as fluorophores. Alternatively, the fluorophores may be bound directly or indirectly to the analytes of interest instead of being bound to the nanowires. In another embodiment, the detection elements 118 may include sensing elements configured to bind with specific analytes of interest in addition or instead of the fluorophores. For example, the sensing elements may include one or more enzymes, proteins, antibodies, or a combination thereof.

Furthermore, for semiconductor nanowires or composite nanowires having light guiding properties, the signal may be increased due to the light guiding properties of the nanowire. For example, the semiconductor nanowire may comprise III-V semiconductor nanowires having Group III-V wires 112, such as III-phosphide wires, e.g., gallium phosphide wires, which have light guiding properties. Furthermore, composite GaP core/silicon oxide shell nanowires may have enhanced light guiding properties due to the silicon oxide shell functioning as an optical cladding for the GaP core.

In various embodiments, at least one end of the nanowires 110 is functionalized with an affinity compound, such as a ligand. In one embodiment, both ends and/or side of the nanowires 110 are functionalized with affinity compounds such as affinity ligands. For example, a first end of the nanowires 110 may be functionalized with a first affinity ligand 120, and a side and/or a second end of the nanowires 110 may be functionalized with a different second affinity ligand 122. The first affinity ligand 120 may be hydrophobic and the second affinity ligand 122 may be hydrophilic, or vice-versa. For example, the first affinity ligand 120 may comprise a thiol ligand, such as 1-octadecanethiol, and the second affinity ligand 122 may comprise a phosphonic acid ligand, such as (12-phosphono dodecyl)phosphonic acid.

In one embodiment, the beads 100 may be formed by a method that includes mixing a liquid containing radiation-curable (e.g., UV-curable) component and a photoinitiator with a liquid containing dispersed nanowires in a dispersion containing two immiscible liquid phases. The mixing of the two liquids results in spheres (i.e., spherical droplets of the first liquid phase) coated with nanowires aligned perpendicular to the sphere surface. In one embodiment, the majority of the nanowire-coated spheres remain floating in the second liquid phase and do not settle and clump at the bottom of the container. It another embodiment, the nanowire-coated spheres do not remain floating. The nanowire-coated spheres are then solidified using radiation (e.g., UV radiation) and/or heat. The formed urchin-like beads can then be collected and transferred to a new solvent for its functionalization with, for instance, sensing elements. This results in a sensor transducer with an enlarged surface area that allows an increase in the interactions between the sensing elements and the sensor transducer resulting in higher signals and/or better sensitivity when detecting analytes of interest.

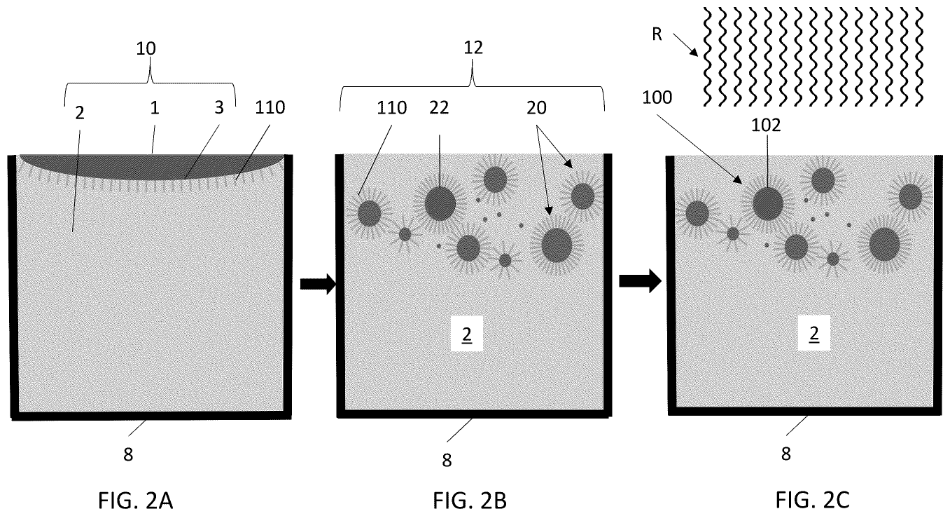

FIGS. 2A-2C illustrate steps of a method of forming urchin like beads 100, according to various embodiments of the present disclosure. As shown in FIG. 2A, a two immiscible liquid phase dispersion is provided in a container 8. The two liquid phases of the dispersion may form a two-phase stratified liquid 10. The stratified liquid 10 may include a first phase 1 and a second phase 2, which may be liquid phases that are separated by an interface 3. The first phase 1 and the second phase 2 may comprises immiscible solvents. In particular, the first phase 1 and the second phase 2 may include different ones of a polar solvent and a non-polar solvent. For example, the second phase 2 may include a polar solvent, such as water, an alcohol, or another hydrophilic solvent. The first phase 1 may include a non-polar solvent, such as a hydrophobic organic solvent. For example, the first phase 1 may include cyclopentanone as a solvent, which is immiscible with water. Other immiscible, partially miscible and/or miscible solvents may be included in the first and the second phases 1, 2.

The first phase 1 may include a polymer precursor. The polymer precursor may include a monomer, such as an acrylate monomer. Exemplary acrylate monomers include trimethylolpropane trimethacrylate, trimethylolpropane ethoxylate triacrylate, tri(propylene glycol) diacrylate, hexanediol dimethacrylate, hexanediol diacrylate, or combinations thereof. Other polymer precursors may also be used.

The first phase 1 may also include a photoinitiator. Exemplary photoinitiators include benzoin isopropyl ether, benzil ketal, acyl-phosphine oxide, α-hydroxy-alkyl-phenone, α-dialkoxy-acetophenone, or α-aminoalkyl-phenone. The photoinitiator is used to subsequently polymerize the monomers in a photoinitiated polymerization process.

The first phase 1 may also include an affinity compound, such as an affinity ligand. Examples of affinity ligands include thiol compounds, for example, 1-octadecanethiol ligands. The ligands may be chemically bound from the first phase 1 to the nanowires 110 to form the affinity ligands 120 which selectively bind to catalyst particles (e.g., gold catalyst particles) 114 located on one end of the nanowires 110 to functionalize the nanowires 110. If desired, a diluent, such as ethanol, may also be added to the first phase 1 to dilute the affinity compound.

The stratified liquid 10 may also include the nanowires 110. The nanowires 110 may be aligned with the interface 3. For example, the nanowires 110 may be aligned with their longest axis substantially perpendicular to the plane of the interface 3. In some embodiments, the nanowires 110 may extend from the first phase 1, through the interface 3, and into the second phase 2.

In some embodiments, one or both opposing ends and/or side of the nanowires 110 may be functionalized with affinity compounds, such as the affinity ligands 120 and/or 122. For example, a first end of the nanowires 110 may be functionalized with the first affinity ligand 120, and at least one of a side and/or an opposing second end of the nanowires may be functionalized with the second affinity ligands 122, as described above.

For example, each of the nanowires 110 may comprise a semiconductor nanowire as described with respect to FIG. 1B that includes the first affinity ligands 120 and/or the second affinity ligands 122. For example, the metal catalyst particles 114 of the nanowires 110 may be functionalized with the first affinity ligands 120 and the semiconductor wires 112 of the nanowires 110 may be functionalized with the second affinity ligands 122, so as to be rendered hydrophobic or hydrophilic.

The affinity compound in the first phase 1 may comprise a compound which has an affinity for the first affinity ligands 120. For example, the metal catalyst particles 114 may be functionalized with a thiol compound, such as 1-octadecanethiol ligands, and the semiconductor wire 112 of the nanowire 110 may be optionally functionalized with the second affinity ligands 122, such as (12-phosphono dodecyl)phosphonic acid ligands. In this example, the affinity compound in the first phase 1 may also comprise a thiol compound, such as 1-octadecanethiol, which has affinity for the 1-octadecanethiol first affinity ligands 120. Other functionalization compounds may also be used.

Due to the functionalization of the nanowires 110, the nanowires 110 align with their longest axis substantially perpendicular to the interface 3. Since the affinity compound in the first phase 1 has an affinity for the first affinity ligands 120, the metal catalyst particles 114 extend from the interface 3 into the first phase 1 (e.g., into the organic/hydrophobic phase), while the semiconductor wires 112 of the nanowires 110 extend from the interface 3 into the second phase 2 (e.g., into the water/hydrophilic phase). The direction of the nanowire alignment at the interface 3 may be reversed if the functionalization compound location is reversed, or the hydrophobicity/hydrophilicity of the functionalization compound(s) or of the first and second phases 1, 2 are reversed.

The stratified liquid 10 may be formed in various ways. In one embodiment, the first and second phases 1, 2 may be provided into the container 8 followed by adding the polymer precursor and the other optional compounds into the stratified liquid 10, such as that the polymer precursor segregates into the first phase 1. Alternatively, the polymer precursor and the other optional compounds are provided into the first phase 1 and followed by combining the first phase 1 and the second phase 2 in the container 8. Furthermore, the first and the second phases 1, 2 may be provided into the container 8 followed by providing the nanowires 110 into the stratified liquid 10. Alternatively, the nanowires 110 may be provided into the first phase 1 or the second phase 2 first, followed by combining the first and second phases 1, 2 together to form the stratified liquid 10. Suitably functionalized nanowires are then prone to assemble at the interface 3 in the container 8, as shown in FIG. 1A.

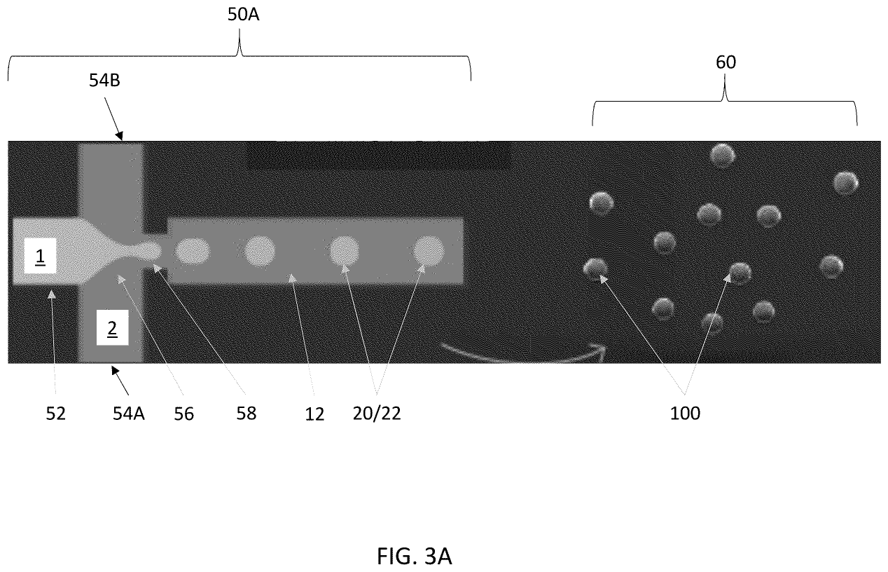

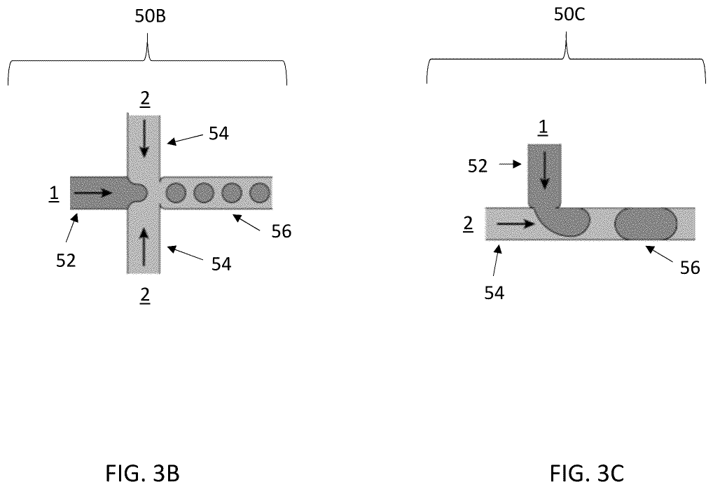

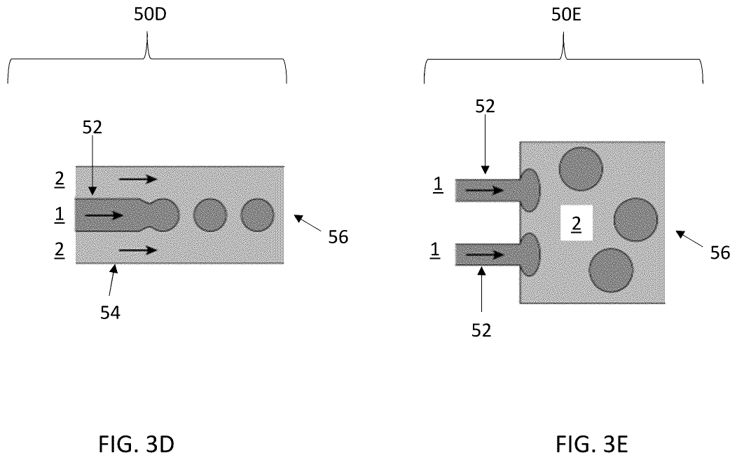

As shown in FIG. 2B, the stratified liquid 10 is agitated to break up the interface 3 and form a dispersion 12. The agitation may be conducted by stirring the stratified liquid 10 with a stirrer, by moving the container 8, by moving the stratified liquid 10, and/or by ultrasonic agitation, to mix the first and second phases 1, 2. The mixing of the first and the second phases 1, 2 and the vertical alignment of the nanowires 110 may be performed simultaneously. Alternatively, the dispersion may be formed in a microfluidic device to decrease polydispersity of the bead size, as will be described below with respect to FIGS. 3A-3E.

As shown in FIG. 2B, the dispersion 12 may include micelles 20 dispersed in the second phase 2. The micelles 20 may comprise droplets/spheres 22 of the first phase 1 surrounded by the nanowires 110. The droplets 22 may include the polymer precursor, the optional photoinitiator, the optional functionalization affinity compound, and/or the optional diluent, dispersed in the first solvent (e.g., in a hydrophobic organic solvent). The functionalization of the nanowires 110 by the ligands may result in the alignment of the nanowires 110, such that the nanowires 110 extend axially from the droplets 22. Specifically, the functionalized metal catalyst particles 114 of the nanowires 110 (see FIG. 1B) contact the surface of the droplets 22, while the semiconductor wires 112 of the nanowires 110 may extend into the second phase 2.

In one embodiment, if the second phase 2 comprises water or an aqueous solution, then the nanowires are dispersed in the water, but the metal catalyst particles 114 (e.g., gold particles) are functionalized with the first affinity ligands 120, which are also present in the droplets 22. In this case, only the metal catalyst particles 114 are disposed within the droplets 22, while the rest of the nanowire (i.e., the semiconductor wires 112) is disposed in the second phase 2. At least 50%, such as at least 70%, such as at least 90% of the droplets 22 in the dispersion 12 are coated with the nanowires 110 and form the micelles 20.

As shown in FIG. 1C, the polymer precursor in the droplets 22 may be polymerized to form the cores 102 of the urchin-like polymer beads 100. In particular, the micelles 20 may be subjected to a polymerization process, such as a photo-polymerization process, in which radiation R, such as UV radiation (or alternatively visible light or IR radiation) is radiated onto the micelles 20. The photo-polymerization may comprise photoinitiated polymerization. Alternatively, the container 8 may be heated to provide thermal polymerization. Thus, the nanowires 110 extending from the droplets 22 become fixed to the cores 102 of the beads 100 during the formation of the cores 102.

The cores 102 may be substantially spherical, solid polymer beads. Multiple nanowires 110 may radially extend, lengthwise, from the cores 102. The metal catalyst particles 114 of the nanowires 110 may be attached to the surfaces of the cores 102 or be embedded in the cores 102. At least 50%, such as at least 70%, such as at least 90% of the surface area of the cores 102 may be coated with the nanowires 110 to form the urchin-like polymer beads 100.

After forming the urchin-like polymer beads 100, the detection elements 118 (see FIG. 1B) may be attached to the beads 100. The detection elements 118 may include enzymes, antibodies, protein, and/or fluorophores. The functionalization of the beads 100 with the detection elements 118 may be performed in the container 8 or in a different container, and may be performed in a different liquid or in the same liquid dispersion 12. For example, the beads 100 may be transferred to a different container containing a different functionalization solution to functionalize the beads 100 with the detection elements 118.

The functionalized beads 100 may be placed in a sensor and used as a sensing element in a sensor for detecting compounds of biomedical relevance or other compounds (e.g. gases). The sensor may comprise an optical sensor. An example of an optical sensor is described in U.S. Patent Application Publication Number US 2017/0212106 A1, published on Jul. 27, 2017 naming Heiner Linke, et. al. as inventors, and incorporated herein by reference in its entirety. The optical sensor may use surface-Raman scattering analyte detection. The optical sensor may include a radiation source (e.g., lamp or laser which emits UV, visible and/or IR radiation), a radiation detector (e.g., photodetector), a processor and the sensor transducers comprising urchin-like beads functionalized with sensing element(s) and optionally with fluorophores. The sensor transducers are placed in contact with an analyte fluid, and are irradiated with radiation from the radiation source. The radiation detector detects changes in radiation that is returned from the sensor transducers if an analyte of interest from the analyte fluid binds to the sensing elements on the sensor transducers. For example, if the fluorophores are initially chemically bound to the nanowires 110, then the analyte may quench the radiation from the fluorophores if the analyte binds to the beads 100. Alternatively, if the fluorophores are initially chemically bound to the analyte of interest, then if the analyte binds to the beads 100 and the fluorophores bound to the analyte emit radiation in response to the radiation from the radiation source. The radiation emitted by the fluorophores is detected by the radiation detector. The processor outputs the results of whether binding of analyte of interest was detected as a display, print out, audio output, etc. In other words, the processor determines if the analyte of interest is present in analyte fluid from the step of detecting changes.

FIG. 3A is a top cross-sectional view of an alternative method of forming the dispersion 12 of FIG. 2B, according to various embodiments of the present disclosure. Referring to FIG. 3A, a microfluidic device 50A may be configured to inject the first phase 1 into the second phase 2, such that the first phase 1 is divided into droplets 22 and dispersed into the second phase 2, thereby forming the dispersion 12. In this embodiment, the container 8 may comprise a portion of the microfluidic device 50A. Specifically, the first phase 1 (e.g., the phase containing the organic solvent and the polymer precursor) is provided into a first inlet 52 of the microfluidic device 50A. The second phase 2 (e.g., water) containing the nanowires is provided into at least one second inlet 54A, 54B of the microfluidic device 50A. The first and second phases 1, 2 are combined in a mixing zone 56 of the microfluidic device 50A to form the stratified liquid as described above with respect to FIG. 2A. The first and second phases 1, 2 are then passed from the mixing zone 56 through a narrow orifice 58 having a smaller size (e.g., diameter) than the mixing zone 56. As a result, micelles 20 and/or droplets 22 are disposed in the second phase 2 to form a dispersion 12, as described above with respect to FIG. 2B. The dispersion 12 then flows to a polymerization zone 60 where the polymer precursor in the micelles 20 is polymerized by radiation (e.g., UV radiation) and/or heat to form the beads 100, as described above with respect to FIG. 2C.

While FIG. 3A illustrates one example of an “X” or “cross” shaped microfluidic device, it should be understood that other microfluidic devices having different configurations and different operating methods may be used to form the dispersion 12. For example flow focusing, T-junction, co-flow or step emulsification microfluidic devices 50B, 50C, SOD or 50E may also be used, as shown in FIGS. 3B, 3C, 3D and 3E, respectively. Such microfluidic devices are described in Hess, David. Yang, Tianjin and Stavrakis, Stavros, Droplet-based optofluidic systems for measuring enzyme kinetics. Analytical and Bioanalytical Chemistry, (2019), 412, 10.1007/s00216-019-02294-z, and in Zhu, Pingan and Wang, Liqiu, Passive and active droplet generation with microfluidics: a review, Lab Chip., (2016) 17, 34-75, 10.1039/C6LC01018K. incorporated herein by reference in their entirety. The first phase 1 is provided into at least one first inlet 52 and the second phase 2 (e.g., water) containing the nanowires is provided into at least one second inlet 54 of these microfluidic devices 50B, 50C, or 50D. In the step emulsification microfluidic device 50E, the first phase 1 is provided into at least one first inlet 52 and the second phase 2 (e.g., water) containing the nanowires which is located in a droplet formation zone 56. Droplets of the first phase 1 are formed in the second phase 2 in a channel or droplet formation zone 56 of these microfluidic devices 50B, 50C 50D or 50E.

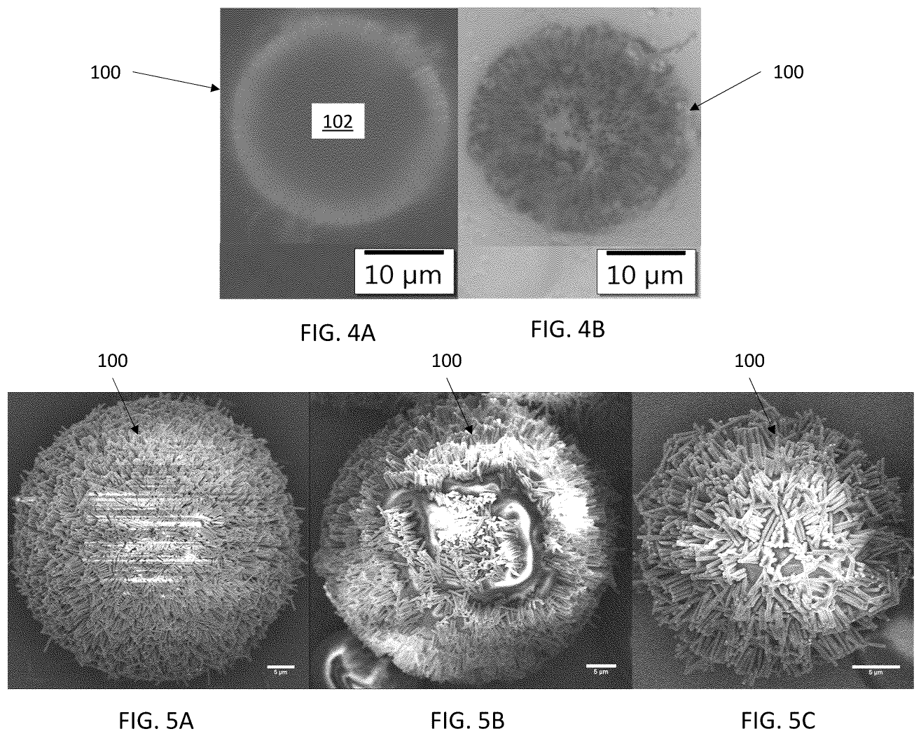

FIGS. 4A and 4B are optical micrographs of the urchin-like beads 100, according to various embodiments of the present disclosure. FIGS. 5A, 5B, and 5C are scanning electron microscopy (SEM) images of urchin-like beads 100, according to various embodiments of the present disclosure. Referring to FIGS. 4A, 4B, and 5A-5C, the beads 100 included solid polymer cores 102 that were nearly completely covered with nanowires 110. As such, the nanowires 110 provided the beads 100 with a high surface area. The nanowires 110 also exhibit a radial orientation with respect to the cores 102.

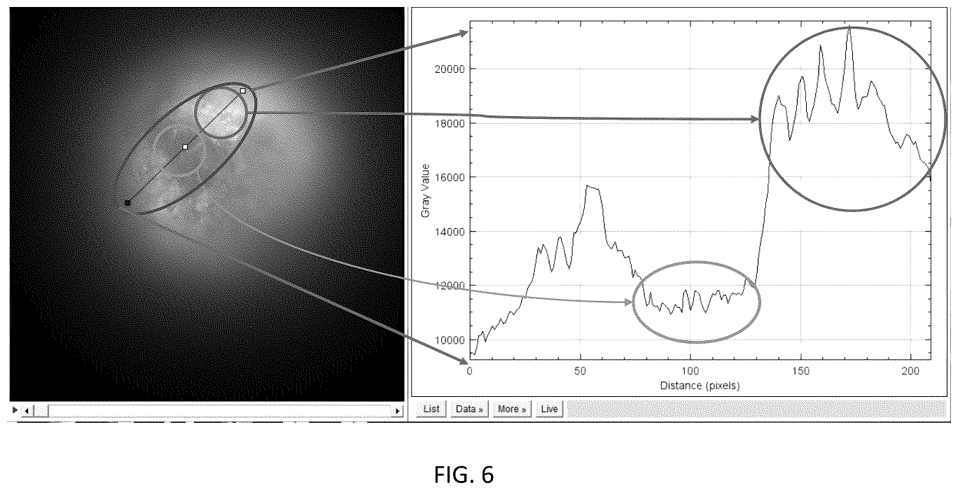

FIG. 6 is a micrograph and chart showing the optical spectrum from an urchin-like bead functionalized with fluorophore molecules, according to various embodiments of the present disclosure. As shown in FIG. 6, areas of the bead with well-aligned nanowires show a higher optical signal intensity than areas without the well-aligned nanowires.

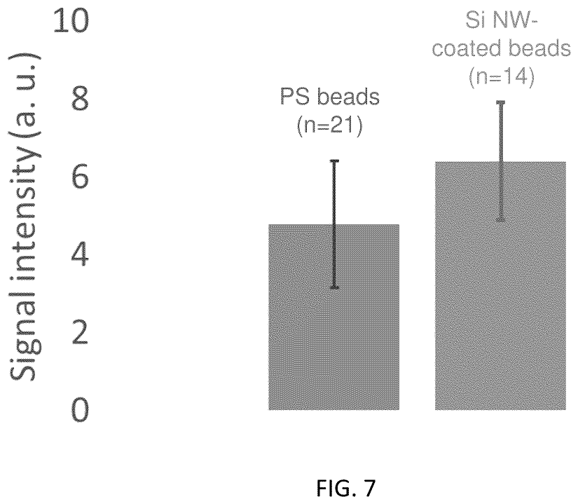

FIG. 7 is a bar graph comparing the spectral intensity from the fluorophore functionalized urchin-like beads containing silicon nanowires according to an embodiment of the present disclosure to comparative fluorophore functionalized polystyrene beads without nanowires. Referring to FIG. 7, the signal intensity is calculated using the following formula:

$$\\ Signal intensity = \frac{( Ibead - Ibackground )}{Ibackground}$$

As shown in FIG. 7, the urchin-like beads provided a 1.3 times higher signal intensity than the polystyrene beads. As such, the nanowires provide a substantial improvement to the signal intensity of the beads.

Although the foregoing refers to particular preferred embodiments, it will be understood that the invention is not so limited. It will occur to those of ordinary skill in the art that various modifications may be made to the disclosed embodiments and that such modifications are intended to be within the scope of the invention. All of the publications, patent applications and patents cited herein are incorporated herein by reference in their entirety.

1. A method, comprising:
forming spherical liquid droplets of a first liquid phase containing a polymer precursor, wherein the spherical liquid droplets are dispersed in a second liquid phase, and wherein nanowires are located at an interface of the first and the second liquid phase and the nanowires extend with their longest axis substantially perpendicular to a surface of the spherical liquid droplets; and
polymerizing the polymer precursor in the spherical liquid droplets to form beads comprising a solid polymer core and multiple nanowires aligned with their longest axis substantially perpendicular to a surface of the solid polymer core.

2. The method of claim 1, wherein:
the first liquid phase further comprises a photoinitiator; and
the polymerizing the polymer precursor comprises exposing the droplets to UV radiation to photopolymerize the polymer precursor.

3. The method of claim 2, wherein the polymer precursor comprises an acrylate-based monomer.

4. The method of claim 3, wherein:
the acrylate-based monomer comprises trimethylolpropane trimethacrylate, trimethylolpropane ethoxylate triacrylate, tri(propylene glycol) diacrylate, hexanediol dimethacrylate, or hexanediol diacrylate; and
the photoinitiator comprises benzoin isopropyl ether, benzil ketal, acyl-phosphine oxide, α-hydroxy-alkyl-phenone, α-dialkoxy-acetophenone, or α-aminoalkyl-phenone.

5. The method of claim 1, wherein the nanowires cover at least 50% of the surface area of each of the solid polymer cores.

6. The method of claim 1, wherein:
the first liquid phase further comprises a non-polar solvent; and
the second liquid phase further comprises a polar solvent.

7. The method of claim 6, wherein at least first ends of the nanowires are functionalized by affinity ligands.

8. The method of claim 6, wherein:
at least one of sides or first ends of the nanowires are functionalized by polar affinity ligands; and
opposing second ends of the nanowires are functionalized by non-polar affinity ligands.

9. The method of claim 8, wherein each of the nanowires comprises:
a metal catalyst particle that comprises the second end of the nanowire and that is functionalized by the non-polar affinity ligands; and
a semiconductor wire that extends from the metal catalyst particle, that comprises the side and the first end of the nanowire and that is functionalized by the polar affinity ligands.

10. The method of claim 9, wherein the first liquid phase further comprises a functionalization affinity compound that has an affinity for the non-polar affinity ligands, such that the metal catalyst particles are disposed in the spherical liquid droplets and the semiconductor wires extend from the spherical liquid droplets and are disposed in the second liquid phase.

11. The method of claim 1, wherein the nanowires comprise semiconductor nanowires, electrically conductive nanowires, dielectric nanowires, or a combination thereof.

12. The method of claim 1, further comprising:
functionalizing the beads with at least one of an enzyme, a protein, an antibody, a fluorescent label, or a combination thereof; and
using the functionalized beads as a sensing element in an optical sensor to detect an analyte having an affinity for the functionalized beads.

13. The method of claim 1, further comprising:
providing the first liquid phase containing the polymer precursor into a microfluidic device;
providing the second liquid phase containing the nanowires into the microfluidic device; and
passing the first liquid phase and the second liquid phase through the microfluidic device to form the spherical liquid droplets of the first liquid phase containing the polymer precursor dispersed in the second liquid phase.

14. A bead comprising:
a solid polymer core; and
nanowires aligned with their longest axis substantially perpendicular to a surface of the solid polymer core.

15. The bead of claim 14, wherein the nanowires comprise semiconductor nanowires, electrically conductive nanowires, dielectric nanowires, or a combination thereof.

16. The bead of claim 14, wherein the nanowires comprise:
a metal catalyst particle attached to the core; and
a semiconductor wire attached to the metal catalyst particle and extending radially from the solid polymer core.

17. The bead of claim 14, wherein the bead is functionalized at least one of an enzyme, a protein, an antibody, a fluorescent label, or a combination thereof.

18. An optical sensor, comprising:
a sensor transducer comprising the bead of claim 17;
a radiation source; and
a radiation detector.

19. An optical sensing method, comprising:
providing an analyte fluid into the optical sensor of claim 18, such that the analyte fluid contacts the sensor transducer;
irradiating the sensor transducer with radiation from the radiation source;
detecting changes in radiation that is returned from the sensor transducer; and
determining if an analyte of interest is present in the analyte fluid based on the detected changes in the radiation.

20. A method, comprising:
forming a dispersion comprising micelles dispersed in a second solvent, the micelles comprising droplets of a first solvent, a polymer precursor dispersed in the first solvent, and nanowires that radially extend from the droplets into the second solvent; and
polymerizing the polymer precursor to form beads that comprise solid polymer cores and the nanowires radially extending from the solid polymer cores.
